(19) United States
(12) Patent Application Publication
(10) Pub. No.: US 2022/0215904 A1
(43) Pub. Date: Jul. 7, 2022

(54) METHODS OF PREDICTING OR VALIDATING THE EFFECTIVENESS OF STACS ON THE BINDING BETWEEN NAD+ AND SIRTUINS

(71) Applicant: Hangzhou Nuoshen Technology Co., Ltd., Hangzhou City (CN)

(72) Inventors: Jun Li, Hangzhou (CN); Yihan Huang, Hangzhou (CN); Xiaosu Tang, Hangzhou (CN)

(21) Appl. No.: 17/317,618

(22) Filed: May 11, 2021

(30) Foreign Application Priority Data
Jan. 5, 2021 (CN) 202110006987.8

(51) Int. Cl. G16C 20/10; G16C 20/30

(52) U.S. Cl. CPC G16C 20/10; G16C 20/30

(57) ABSTRACT

The present disclosure relates to a method of a method of predicting or validating the effectiveness of STACs on the binding between nicotinamide adenine dinucleotide (NAD⁺) and sirtuins.

### CLAIM OF PRIORITY

This application claims the benefit of Chinese Patent Application App. No. 202110006987.8, filed on Jan. 5, 2021. The entire content of the foregoing application is incorporated herein by reference.

### TECHNICAL FIELD

The disclosure relates to the field of dietary supplements and biochemistry, in particular to methods of predicting or validating the effectiveness of STACs on the binding between NAD⁺ and sirtuins.

### BACKGROUND

Mammalian sirtuins are nicotinamide adenine dinucleotide (NAD⁺)-dependent deacylases that regulate multiple cellular functions including cell survival, mitochondrial biogenesis, inflammation, aging, circadian rhythms, stress resistance, energy efficiency, and alertness during low-calorie situations. There are seven sirtuins (i.e., SIRT1-SIRT7) in mammals that occupy different subcellular compartments with various functions. For example, SIRT1, SIRT6, and SIRT7 are predominantly located in the nucleus, and regulate metabolism, DNA repair, rRNA transcription, and inflammation; SIRT2 is predominantly located in cytoplasm, and regulates cell cycle and tumorigenesis; SIRT3, SIRT4, and SIRT5 are predominantly located in mitochondria and regulate metabolism, insulin secretion, and ammonia detoxification. During the aging process, the in vivo level of NAD⁺ declines. NAD⁺ depletion affects sirtuins' activities, which forms a negative feedback loop, and eventually causes aging-related diseases. Therefore, the sirtuin family, with its ability to extend human lifespan, has become one of the hottest research topics. Many compounds have been discovered to activate sirtuins and enhance the interaction between NAD⁺ and sirtuins. Resveratrol, a type of natural phenol, is the first found sirtuin-activating compound (STAC) that effectively activates SIRT1, and has been proved to extend the lifespan of yeast and other simple organisms. SIRT1-activating compounds, e.g., resveratrol, act as a SIRT1 allosteric activator. Specifically, the SIRT1-activating compounds can bind to the N-terminal domain (NTD) of SIRT1 and facilitate the interaction between the NTD and the catalytic domain (CD) of SIRT1 via a “bend-at-the-elbow” model (See Kane, A. E., et al. “Pharmacological Approaches for Modulating Sirtuins.” Introductory Review on Sirtuins in Biology, Aging, and Disease. Academic Press, 2018. 71-81), thereby increasing the binding affinity of a substrate for SIRT1. Moreover, resveratrol can be found in natural foods such as grapes, blueberries, and cranberries. Although many researches have shown the effectiveness of resveratrol as a SIRT1-activating compound to increase lifespan and resveratrol has been widely used as a dietary supplement, its binding mechanism with other sirtuins and the corresponding effectiveness to longevity are still unclear, let alone other polyphenolic STACs that are less studied. (See Kane, A. E., et al. “Sirtuins and NAD⁺ in the development and treatment of metabolic and cardiovascular diseases.” Circulation Research 123.7 (2018): 868-885).

Resveratrol is a polyphenolic compound. In general, dietary polyphenols can be categorized into four subclasses according to their chemical structures: flavonoids, phenolic acids, stilbenes, and lignans. Resveratrol, pterostilbene, hesperatin, naringenin, catechin, quercetin, fisetin, caffeic acid, pinoresinol, and pyrroloquinoline quinone (PQQ) are all natural polyphenols. These compounds are plant-based antioxidants and can be used as dietary supplements. Therefore, they are considered natural STACs. However, their binding mechanisms with sirtuins and activation effectiveness are not known.

### SUMMARY

The conventional experimental schemes to screen effective STACs have several drawbacks. For example, the screening process is time-consuming; the procedures are relatively complicated; and the screening is usually insufficient under limited time frame and labor resources. To overcome this problem, molecular dynamics (MD) simulations can be used to determine the binding sites of a particular STAC and/or NAD⁺ on a sirtuin protein, the conformational change, the stability change, and the binding free energy change from a molecular perspective, thereby assisting the STAC screening process. Conventional MD simulations are usually performed to assist prediction or validation process to study the interaction between a ligand molecule and a corresponding receptor protein. For example, MD simulations can be performed to obtain the binding information between a single STAC ligand and the sirtuin receptor system, and then in vitro or in vivo experiments can be performed to verify the effect on NAD⁺ binding to the system. Considering the possibility that binding of the STAC to sirtuin may interfere the binding of NAD⁺ to the sirtuin, the conventional MD simulations may face the difficulty of not sampling through the whole free energy space such that the STAC/Sirtuin/NAD⁺ complex cannot escape from the local minimum to reach the most stable conformation, which could lead to inaccurate results for binding free energy and stability determination.

In view of the above technical problems and the deficiencies in the field, the present disclosure provides methods of predicting or validating the effectiveness of STACs on the binding between NAD⁺ and sirtuins, which enables the STAC/Sirtuin/NAD⁺ complex to reach global minimum in the free energy space and achieve its most stable conformation, via a series of special and rigorous treatments and replica-exchange molecular dynamics simulations. In addition, the methods described herein can be used to predict and/or validate the effectiveness of the STACs computationally by analyzing the stability and binding free energy on the fully equilibrated and stabilized STAC/Sirtuin/NAD⁺ complexes. Prior to the present disclosure, no similar methods and procedures have been reported in the field of dietary supplements, especially in the field of predicting and/or validating the effective of STACs in STAC/Sirtuin/NAD⁺ co-ligand complexes.

In one aspect, the disclosure is related to a method of predicting or validating the effectiveness of a STAC on the binding between NAD⁺ and a sirtuin protein, characterized in that the method includes a replica-exchange molecular dynamics simulation, the method comprising:

(1) obtaining the structural data of a sirtuin protein from Protein Data Bank;

(2) generating the molecular structural input files for a STAC candidate and NAD⁺ using a molecular visualization software;

(3) docking the NAD⁺ to the corresponding binding pocket of the sirtuin protein (e.g., a sirtuin of choice) to obtain a Sirtuin/NAD⁺ complex structure; docking the STAC candidate (e.g., a STAC candidate ligand of choice) to the corresponding binding pocket of the Sirtuin/NAD⁺ complex structure to obtain a STAC/Sirtuin/NAD⁺ complex structure; and leaving the Sirtuin/NAD⁺ complex structure as a control;

(4) generating the topology files, prmtop files, and inperd files of the ligand, receptor, and complex system of both the Sirtuin/NAD+ complex and STAC/Sirtuin/NAD⁺ complex;

(5) converting the topology files in step (4) to Gromacs format;

(6) performing the replica-exchange molecular dynamics simulation on the two complex systems, comprising (preferably in a time order): performing a first round of energy minimization to both systems, respectively; solvating the systems and adding Na⁺ and Cl⁻ to achieve charge neutralization; performing a first round of molecular dynamics simulation in canonical ensemble to the acquired solvated and charge neutralized systems; performing a second round of energy minimization; performing a second round of molecular dynamics simulation in canonical ensemble and a molecular dynamics simulation in isothermal-isobaric ensemble until the systems are fully equilibrated; performing the replica-exchange molecular dynamics simulation to the equilibrated systems; obtaining the stable conformation structures of both the Sirtuin/NAD⁺ and STAC/Sirtuin/NAD⁺ complexes from the free energy space minima; and obtaining the corresponding trajectory files;

(7) removing the solvents from the trajectory files obtained in step (6); performing Cα RMSD calculation and RMSF calculation to determine if the STAC candidate stabilizes the Sirtuin/NAD⁺ complex;

(8) extracting snapshots at certain frequency (e.g., every 1 ns, every 2 ns, every 3 ns, every 4 ns, every 5 ns, every 6 ns, every 7 ns, every 8 ns, every 9 ns, every 10 ns, or every 20 ns) along the no-solvent trajectories from step (7), and performing binding free energy calculation between NAD⁺ and the sirtuin protein for both complexes to determine if the STAC candidate improves the binding between NAD⁺ and the sirtuin protein;

(9) predicting or validating the influence of the STAC candidate to the Sirtuin/NAD⁺ complex, according to stability changes (e.g., the Cα RMSD, RMSF changes) and binding free energy changes observed in step (7) and step (8), respectively.

In some embodiments, the methods described herein also include an additional step. In some embodiments, the additional step comprises administering an effective amount of the STAC candidate to a subject (e.g., a human patient) in need thereof. In some embodiments, the human patient has a cancer. In some embodiments, the additional step comprises verifying the effect of the STAC candidate using in vitro or in vivo assays.

In some embodiments, the STAC described herein is a polyphenolic STAC. In some embodiments, the STAC candidate described herein is a polyphenolic STAC candidate.

In some embodiments, “predicting or validating” is just for polyphenolic STACs. For polyphenolic STAC candidates whose effectiveness are unknown, the disclosure provides methods to accurately predict their effectiveness. For polyphenolic STACs that are known to have effect, the disclosure provides methods to accurately validate their effectiveness.

In some embodiments, the methods described herein utilize replica-exchange molecular dynamics simulations that prevent the system from being trapped in the local minimum of the free energy space, which helps to acquire stable conformations of the Sirtuin/NAD⁺ complex and/or STAC/Sirtuin/NAD⁺ complex more accurately.

In some embodiments, the methods described herein improve the accuracy of the prediction and/or validation of the effect of the STAC candidate on the binding of NAD⁺ to the sirtuin protein, via a series of special and rigorous treatments and an optimized workflow.

In some embodiments, in step (2), the molecular visualization software is VMD (Visual Molecular Dynamics, University of Illinois), PyMol (the PyMOL Molecular Graphics System, Version 2.0 Schrödinger, LLC.), or any other similar software. Details of VMD can be found, e.g., in Humphrey, W., et al., “VMD—Visual Molecular Dynamics”, J. Molec. Graphics, 1996, vol. 14, pp. 33-38, which is incorporated herein by reference in its entirety.

In some embodiments, in step (4), the files are generated using AmberTools.

In some embodiments, in step (6), the force field used for the replica-exchange molecular dynamics simulations is selected from the group consisting of AmberFF14S, Amber99S, gromacs54a, GROMOS9, and GAFF.

In some embodiments, in step (6), energy minimization is carried out using steepest descents algorithm until the maximum force is no greater than 2000 kJ/mol/nm, no greater than 1500 kJ/mol/nm, no greater than 1400 kJ/mol/nm, no greater than 1300 kJ/mol/nm, no greater than 1200 kJ/mol/nm, no greater than 1100 kJ/mol/nm, no greater than 1000 kJ/mol/nm, no greater than 900 kJ/mol/nm, no greater than 800 kJ/mol/nm, no greater than 700 kJ/mol/nm, no greater than 600 kJ/mol/nm, or no greater than 500 kJ/mol/nm.

In some embodiments, in step (6), the minimum distance between the solutes and the edge of the simulation box is no less than 5 nm, no less than 4 nm, no less than 3 nm, no less than 2 nm, no less than 1 nm, or no less than 0.5 nm. In some embodiments, the water model is SPC/E or TIP3P.

In some embodiments, in step (6), the canonical ensemble molecular dynamics simulations are performed under periodic boundary conditions, and the first round of canonical ensemble molecular dynamics simulation further comprises of heating the systems to 300-320 K (e.g., about 300 K, about 301 K, about 302 K, about 303 K, about 304 K, about 305 K, about 306 K, about 307 K, about 308 K, about 309 K, about 310 K, about 311 K, about 312 K, about 313 K, about 314 K, about 315 K, about 316 K, about 317 K, about 318 K, about 319 K, or about 320 K) in less than 20 picosecond (e.g., less than 20 ps, less than 19 ps, less than 18 ps, less than 17 ps, less than 16 ps, less than 15 ps, less than 14 ps, less than 13 ps, less than 12 ps, less than 11 ps, less than 10 ps, less than 9 ps, less than 8 ps, less than 7 ps, less than 6 ps, less than 5 ps, less than 4 ps, less than 3 ps, less than 2 ps, or less than 1 ps) to release extra internal strain with a timestep less than 1 femtosecond; the second round of canonical ensemble molecular dynamics simulation further comprises heating and running the system at 300-320 K (e.g., about 300 K, about 301 K, about 302 K, about 303 K, about 304 K, about 305 K, about 306 K, about 307 K, about 308 K, about 309 K, about 310 K, about 311 K, about 312 K, about 313 K, about 314 K, about 315 K, about 316 K, about 317 K, about 318 K, about 319 K, or about 320 K) for at least 50 picosecond (e.g., at least 50 ps, at least 55 ps, at least 60 ps, at least 65 ps, at least 70 ps, at least 75 ps, at least 80 ps, at least 90 ps, at least 100 ps, at least 150 ps, at least 200 ps, at least 250 ps, at least 300 ps, at least 350 ps, at least 400 ps, at least 450 ps, at least 500 ps, at least 600 ps, at least 700 ps, at least 800 ps, at least 900 ps, or at least 1 ns) with a timestep greater than 1 femtosecond (e.g., greater than 2 ns, greater than 3 ns, greater than 4 ns, greater than 5 ns, greater than 6 ns, greater than 7 ns, greater than 8 ns, greater than 9 ns, or greater than 10 ns). In some embodiments, temperature is set to be about 310 K to mimic human body temperature. The short first round of canonical ensemble molecular dynamics simulation can eliminate the excess unphysical contact between solutes and solvent for better equilibration in the next steps.

In some embodiments, in step (6), the isothermal-isobaric molecular dynamics simulation is performed under periodic boundary condition. In some embodiments, temperature is controlled to be about 300-320 K (e.g., about 300 K, about 301 K, about 302 K, about 303 K, about 304 K, about 305 K, about 306 K, about 307 K, about 308 K, about 309 K, about 310 K, about 311 K, about 312 K, about 313 K, about 314 K, about 315 K, about 316 K, about 317 K, about 318 K, about 319 K, or about 320 K) using Velocity Rescale (temperature coupling using velocity rescaling with a stochastic term; See Bussi, G., et al. “Canonical sampling through velocity rescaling.” The Journal of Chemical Physics 126.1 (2007): 014101.) and pressure is controlled to be about 1 atm Parinello-Rahman (extended ensemble pressure coupling where the box vectors are subject to an equation of motion; See Parrinello, M. et al. “Polymorphic transitions in single crystals: A new molecular dynamics method.” Journal of Applied Physics 52.12 (1981): 7182-7190), and the systems are equilibrated for at least 50 picoseconds. In some embodiments, the temperature is set to be about 310 K.

In some embodiments, in step (6), the replica-exchange molecular dynamics simulation is a temperature replica-exchange molecular dynamics simulation, and the temperature is set to be about 300-500 K (e.g., about 300 K, about 310 K, about 320 K, about 330 K, about 340 K, about 350 K, about 400 K, about 450 K, or about 500 K). In some embodiments, in step (6), the replica-exchange molecular dynamics simulation is a Hamilton replica-exchange molecular dynamics simulation, and the temperature is set to be any single value in the range of 300-500 K (e.g., about 300 K, about 310 K, about 320 K, about 330 K, about 340 K, about 350 K, about 400 K, about 450 K, or about 500 K). In some embodiments, the temperature is set to be 310 K.

In some embodiments, in step (6), all water bonds are constrained with SETTLE, and all other bonds are constrained with LINCS.

In some embodiments, in step (6), a 5 nm cutoff, 4 nm cutoff, 3 nm cutoff, 2 nm cutoff, 1 nm cutoff, 0.5 nm cutoff, or 0.1 nm cutoff is used for short range non-bonded interactions. In some embodiments, Particle Mesh Ewald (PME) method is used for long-range electrostatics calculations (See Darden, T., et al. “Particle mesh Ewald: An N·log (N) method for Ewald sums in large systems.” The Journal of Chemical Physics 98.12 (1993): 10089-10092).

In some embodiments, in step (9), by evaluating the RMSD calculation results obtained in step (7), if the overall RMSD of the STAC/Sirtuin/NAD⁺ complex is smaller than 1 nm and is smaller than the overall RMSD of the Sirtuin/NAD⁺ complex, the STAC candidate stabilizes the Sirtuin/NAD⁺ complex.

In some embodiments, in step (9), by evaluating the RMSF calculation results obtained in step (7), if the RMSF values of the binding site residues of the STAC candidate on the sirtuin of choice are smaller than 1 nm, the binding site of the STAC candidate on the sirtuin protein is stable, if in the STAC/Sirtuin/NAD⁺ complex the RMSF values of the binding site residues of NAD⁺ on the sirtuin of choice are smaller than 1 nm, the STAC candidate makes the binding between NAD⁺ and the sirtuin protein more stable.

In some embodiments, in step (9), by evaluating the binding free energy calculation results obtained in step (8), if the binding free energy ΔG in the STAC/Sirtuin/NAD⁺ complex is negative, and its absolute value is greater than that in the Sirtuin/NAD⁺ complex, adding the STAC candidate strengthens the binding between NAD⁺ and the sirtuin protein.

The method described in the present disclosure is applicable of predicting and/or validating the STAC candidates with known or unknown effectiveness and all the seven sirtuin proteins (e.g., SIRT1, SIRT2, SIRT3, SIRT4, SIRT5, SIRT6, and SIRT7).

In some embodiments, the STAC candidate is selected from flavonoids, phenolic acids, stilbenes, and lignans. In some embodiments, the STAC candidate is selected from any one of resveratrol, pterostilbene, hesperatin, naringenin, catechin, quercetin, fisetin, caffeic acid, pinoresinol, pyrroloquinoline quinon, pycnogenol, curcumin, and jaceosidin.

In some embodiments, the sirtuin protein is chosen from any one of human SIRT1, SIRT2, SIRT3, SIRT4, SIRT5, SIRT6, and SIRT7.

The value of the present disclosure is providing a computational method that predicts and/or validates the effectiveness of STACs among dietary supplements from molecular scale and a perspective of mechanism.

In one aspect, the disclosure further provides a method of predicting or validating the effectiveness of a sirtuin-activating compounds (STAC) on the binding between NAD+ and a sirtuin protein, wherein the method comprises a replica-exchange molecular dynamics simulation, the method comprising:

(1) obtaining structural data of a sirtuin protein;

(2) generating a molecular structure for a STAC candidate and NAD+;

(3) docking the NAD+ to the corresponding binding pocket of the sirtuin protein to obtain a Sirtuin/NAD+ complex structure; docking the STAC candidate to the corresponding binding pocket of the Sirtuin/NAD+ complex structure to obtain a STAC/Sirtuin/NAD+ complex structure; and leaving the Sirtuin/NAD+ complex structure as a control;

(4) generating complex systems of both the Sirtuin/NAD+ complex and STAC/Sirtuin/NAD+ complex;

(5) performing the replica-exchange molecular dynamics simulation on the two complex systems;

(6) performing Cα RMSD calculation and RMSF calculation to determine if the STAC candidate stabilizes the Sirtuin/NAD+ complex;

(7) extracting snapshots at a frequency along the no-solvent trajectories, and performing binding free energy calculation between NAD+ and the sirtuin protein for both complexes to determine if the STAC candidate improves the binding between NAD+ and the sirtuin protein; and

(8) predicting or validating the effect of the STAC candidate to the Sirtuin/NAD+complex, according to the Cα RMSD, RMSF, and/or binding free energy changes.

In some embodiments, the replica-exchange molecular dynamics simulation includes one or more of the following steps: performing a first round of energy minimization to both systems, respectively; solvating the systems and adding Na+ and Cl− to achieve charge neutralization; performing a first round of molecular dynamics simulation in canonical ensemble to the acquired solvated and charge neutralized systems; performing a second round of energy minimization to both systems, respectively; performing a second round of molecular dynamics simulation in canonical ensemble and a molecular dynamics simulation in isothermal-isobaric ensemble until the systems are fully equilibrated; performing the replica-exchange molecular dynamics simulation to obtain equilibrated systems; obtaining the stable conformation structures of both the Sirtuin/NAD+ and STAC/Sirtuin/NAD+ complexes from the free energy space minima.

In some embodiments, the method further comprises performing one or more experiments for testing effectiveness of a sirtuin-activating compounds (STAC) on the binding between NAD+ and a sirtuin protein. In some embodiments, the method further comprises administering an effective amount of the STAC candidate to a subject in need thereof.

Compared with the existing technologies, the main advantages of the present disclosure are described below.

(1) The equilibration steps including multiple energy minimizations and canonical ensemble molecular dynamics simulations are more rigorous as compared to existing technologies, which makes the complex systems better equilibrated and easier to reach the most stable conformations. As a result, the complex stabilities, local residue stabilities, and the binding free energies are more accurate as compared to those determined by the existing technologies.

(2) The present disclosure overcomes the defects of the existing technologies in the inaccuracy of treating co-ligand and receptor system. The replica-exchange molecular dynamics simulations proposed in the present disclosure can facilitate the STAC/Sirtuin/NAD⁺ complex find its most stable conformation by searching for the global minimum in the free energy space, such that the complex stabilities, local residue stabilities, and the binding free energies that rely on the conformational structure are more accurate.

(3) The present disclosure has a wide range of applicable objects. In fact, all certified dietary polyphenols that have potential effect to human sirtuin proteins can be selected as the objects of this method. Their effectiveness as STACs can be predicted prior to experiments (e.g., in vitro or in vivo experiments), thereby increasing the efficiency of screening the effective STACs.

(4) The present disclosure can be applied to validate the mechanisms of effective STACs and binding schemes from a molecular perspective.

(5) The present disclosure can be widely applied in the field of dietary supplements where the interaction between proteins and ligands are important.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows the workflow of the method of predicting or validating the effectiveness of STACs on the binding between NAD⁺ and sirtuin.

FIG. 2 shows the Cα RMSD comparison between the SIRT1/NAD⁺ complex and the pterostilbene/SIRT1/NAD⁺ complex.

FIG. 3 shows the RMSF comparison between the SIRT1/NAD⁺ complex and the pterostilbene/SIRT1/NAD⁺ complex.

FIG. 4 shows the Cα RMSD comparison between the SIRT3/NAD⁺ complex and the PQQ/SIRT3/NAD⁺ complex.

FIG. 5 shows the RMSF comparison between the SIRT3/NAD⁺ complex and the PQQ/SIRT3/NAD⁺ complex.

### DETAILED DESCRIPTION

The present disclosure relates to methods of predicting and/or validating the effectiveness of STACs on the binding between nicotinamide adenine dinucleotide (NAD⁺) and sirtuins. The methods include using a series of special and rigorous treatments to complex systems and using replica-exchange molecular dynamics simulations to achieve the global optimization of the complex systems to obtain the most stable conformations of the systems. The complex stabilities, residue stabilities, and binding free energies obtained from these systems can be used to predict and/or validate the effectiveness of STACs on the binding between NAD⁺ and sirtuins. Compared to experimental schemes, the present disclosure has lower time and labor costs, which makes the screening process for effective STACs more efficiently. Compared to conventional computational schemes, the present disclosure provides methods that can identify stable conformation of protein/co-ligand complex more accurately, which further enables more accurate stability and binding free energy calculations with improved efficiency.

The following is a further explanation of the disclosure with reference to the drawings and specific embodiments. It is to be understood that the following embodiments are only used to illustrate the disclosure, but not used to limit the application scope of the present disclosure. For the operation methods whose specific conditions are not indicated in the following embodiments, the standard conditions or the conditions recommended by the manufacturer is to be taken.

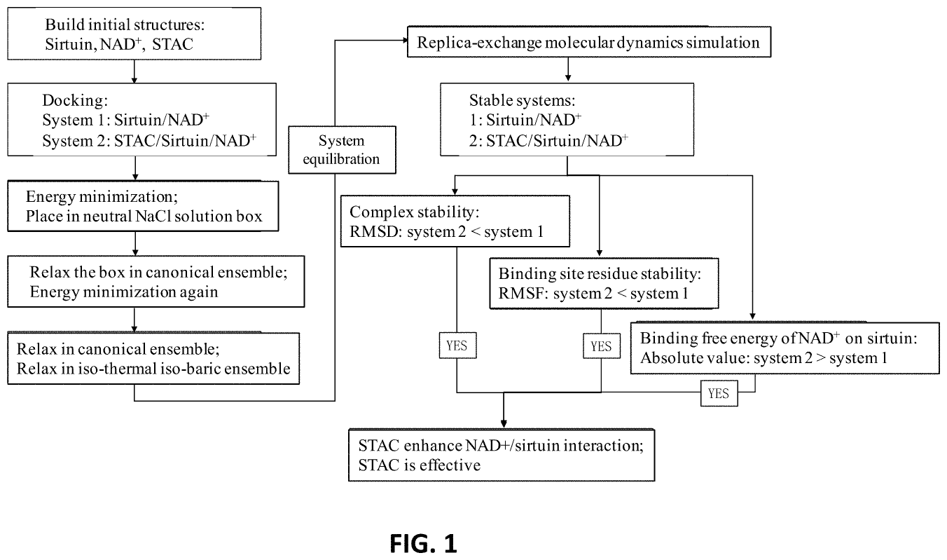

Provided herein are methods for predicting and/or validating the effectiveness of STACs on the binding between NAD⁺ and sirtuins. An exemplary workflow of the methods is shown in FIG. 1. Specifically, the methods comprise the following steps:

Step 1. building the initial structures and initial files of the sirtuin protein, NAD⁺, and the STAC (e.g., a polyphenol candidate);

Step 2. docking to obtain:
System 1: Sirtuin/NAD⁺,
System 2: STAC/Sirtuin/NAD⁺;

and performing the following Steps 3-6 to both System 1 and System 2;

Step 3. placing into charge neutralized NaC1 and water boxes and performing energy minimization;

Step 4. relaxing the above boxes in canonical ensemble, and then performing energy minimization again;

Step 5. relaxing the above boxes in canonical ensemble, and then in isothermal-isobaric ensemble, until the boxes are fully equilibrated;

Step 6. performing replica-exchange molecular dynamics simulation to the equilibrated System 1 and System 2 to obtain the stable conformation of System 1 and System 2, respectively; and

Step 7. comparing the stable System 1 and 2 in Step 6 for the complex stability, NAD⁺ binding site residue stability, and the binding free energy between NAD⁺ and the sirtuin protein, and predicting and/or validating the effectiveness of STACs on the binding between NAD⁺ and sirtuins.

### Molecular Dynamics Simulation

Molecular dynamics (MD) is a computer simulation method for analyzing the physical movements of atoms and molecules. The atoms and molecules are allowed to interact for a fixed period of time, giving a view of the dynamic “evolution” of the system. In the most common version, the trajectories of atoms and molecules are determined by numerically solving Newton's equations of motion for a system of interacting particles, where forces between the particles and their potential energies are often calculated using interatomic potentials or molecular mechanics force fields. The method is applied mostly in chemical physics, materials science, and biophysics.

Some commonly used tools for MD simulation and related to MD simulation are also disclosed. For example, Gromacs (GROningen MAchine for Chemical Simulations, University of Groningen) is a molecular dynamics package mainly designed for simulations of proteins, lipids, and nucleic acids, and GOLD (Genetic Optimisation for Ligand Docking, Cambridge Crystallographic Data Centre) is a genetic algorithm for docking flexible ligands into protein binding sites. Details of Gromacs and GOLD and their applications can be found, e.g., in Bekker, H., et al. “Gromacs-a parallel computer for molecular-dynamics simulations.” 4th International Conference on Computational Physics (PC 92). World Scientific Publishing, 1993; and Jones, G., et al. “Development and validation of a genetic algorithm for flexible docking.” Journal of Molecular Biology 267.3 (1997): 727-748, respectively; each of which is incorporated herein by reference in its entirety.

Additional tools include MMPBSA and MMGBSA (details of MMPBSA and MMGBSA can be found. e.g., in Srinivasan, J, et al. “Continuum solvent studies of the stability of DNA, RNA, and phosphoramidate—DNA helices.” Journal of the American Chemical Society 120.37 (1998): 9401-9409; and Still, W. C., et al. “Semianalytical treatment of solvation for molecular mechanics and dynamics.” Journal of the American Chemical Society 112.16 (1990): 6127-6129). Each of the forgoing articles is incorporated herein by reference in its entirety.

### Methods of Screening

Included herein are methods for screening STACs, e.g., natural STACs or un-natural STACs, by in vitro or in vivo assays, to confirm the prediction and/or validation results (e.g., whether a STAC can stabilize the Sirtuin/NAD⁺ complex, or whether a STAC can improve the binding between NAD⁺ and the sirtuin protein) obtained using the methods described herein. In some embodiments, the in vitro assays can be any of the assays used to determine binding affinities between molecules (e.g., ligand-receptor binding assays), e.g., a ligand binding assay (LBA). In some embodiments, the in vitro assays are used to determine the presence and extent of the ligand-receptor complexes formed, e.g., electrochemically or through a fluorescence detection method. In some embodiments, the in vitro assays are radioligand assays. In some embodiments, the in vitro assays are non-radioactive binding assays, e.g., assays using fluorescence polarization (FP), fluorescence resonance energy transfer (FRET), or surface plasmon resonance (SPR). In some embodiments, the in vitro assays are liquid phase binding assays, e.g., immunoprecipitation (IP). In some embodiments, the in vitro assays are solid phase binding assays, e.g., assays using multiwall plates, on-bead binding, or on-column binding. In some embodiments, the in vitro assays are competitive binding assays. In some embodiments, the in vivo assays described herein are cell-based assay. In some embodiments, the screening as described herein is a high-throughput screening.

In some embodiments, the STAC or STAC candidate is a small molecule. As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

### Methods of Treatment

The methods described herein include methods for the treatment of disorders associated with metabolic and cardiovascular diseases. Generally, the methods include administering a therapeutically effective amount of the STAC or STAC candidate as described herein, to a subject who is in need of, or who has been determined to be in need of, such treatment. In some embodiments, the STAC or STAC candidate described herein can be used as a dietary supplement. In some embodiments, the subject is a model animal, e.g., a mouse. In some embodiments, the subject is a human patient.

### EXAMPLES

The invention is further described in the following examples, which do not limit the scope of the invention described in the claims.

### Example 1: Validation of the effectiveness of Pterostilbene on NAD⁺/SIRT1 Binding

According to the above methods and procedures, experiments were carried out to validate the effectiveness of pterostilbene on the binding between NAD⁺ and SIRT1. The results confirmed that pterostilbene is an effective SIRT1-activating compound. Detailed steps are described as follows.

The SIRT1 structural data was extracted from Protein Data Bank (PDB), i.e., 5BTR.pdb, and the resveratrol molecules in 5BTR.pdb were removed to obtain the SIRT1 structural data. The PDB file of NAD⁺ was extracted from 4IF6.pdb. In addition, the partial charges were calculated and special structural information of pterostilbene molecule was obtained using Gaussian (computational chemistry software package, Gaussian 16, Revision C.01, Gaussian, Inc., Wallingford Conn., 2016). All the structural information above were combined to create the structural input file from VMD.

Next, GOLD (See Jones, G., et al. “Development and validation of a genetic algorithm for flexible docking.” Journal of molecular biology 267.3 (1997): 727-748) was used for the docking of NAD⁺ and pterostilbene onto SIRT1. Docking scores were obtained and compared. Both the SIRT1/NAD⁺ complex structure and pterostilbene/SIRT1/NAD⁺ complex structure were obtained.

Next, AmberTools (See Case, D. A., et al. “Amber 2020.” (2020)) was used to generate the topology files, prmtop (parameter/topology file specification) files, and inperd (coordinate file specification) files for the complexes, SIRT1, pterostilbene, and NAD⁺ in both the SIRT1/NAD⁺ complex structure and pterostilbene/SIRT1/NAD⁺ complex structure. The topology files, prmtop files, and inperd files are output files from AmberTools and used in Gromacs. Acpype.py (See Da S., et al. “ACPYPE-Antechamber python parser interface.” BMC research notes 5.1 (2012): 1-8) was then used to convert the topology files to Gromacs formats. A temperature replica-exchange molecular dynamics simulation was then performed using the GAFF force field (See Wang, J., et al. “Development and testing of a general amber force field.” Journal of Computational Chemistry 25.9 (2004): 1157-1174). All water bonds are constrained with SETTLE (See Miyamoto, S., et al. “Settle: An analytical version of the SHAKE and RATTLE algorithm for rigid water models.” Journal of Computational Chemistry 13.8 (1992): 952-962), and all other bonds are constrained with LINCS (See Hess, B., et al. “LINCS: a linear constraint solver for molecular simulations.” Journal of Computational Chemistry 18.12 (1997): 1463-1472).

In Gromacs, a first round of energy minimization was performed to the SIRT1/NAD⁺ complex and pterostilbene/SIRT1/NAD⁺ complex via steepest descents until the maximum force is no greater than 1000 kJ/mol/nm. Then, the above systems were solvated in rectangular SPC/E (See Berendsen, H. J. C., et al. “The missing term in effective pair potentials.” Journal of Physical Chemistry 91.24 (1987): 6269-6271) water boxes. Na⁺ and Cl⁻ ions were added to achieve charge neutralization to get 0.155 M NaCl-complex systems, and the solutes were required to be at least 1.2 nm away from the edges of the boxes. Afterwards, a first round of molecular dynamics simulation in canonical ensemble was performed, followed by a second round of energy minimization, with Velocity Rescale to heat the systems to and at 310 K for 10 ps (timestep=0.2 fs). Next, the second round of molecular dynamics simulation in canonical ensemble was performed for 100 ps (timestep=2 fs) and a molecular dynamics simulation in isothermal-isobaric ensemble was performed for 100 ps (timestep=2 fs) with Velocity Rescale for temperature control and Parinello-Rahman for pressure control, until temperature is stabilized at 310 K and pressure is stabilized at 1 atm, and the systems were fully equilibrated. Then, 20 replicas at different temperatures in the range of 310 K-400 K for both equilibrated complex systems were created, and a 100 ns temperature replica-exchange molecular dynamics simulation was performed to obtain the stable conformational structures corresponding to the lowest energy for both the SIRT1/NAD⁺ complex and pterostilbene/SIRT1/NAD⁺ complex with their traj ectories.

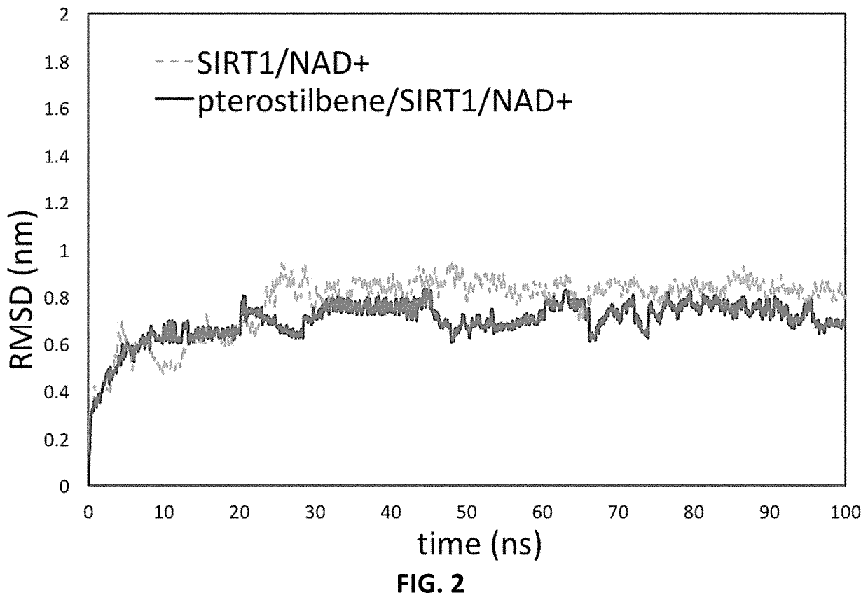

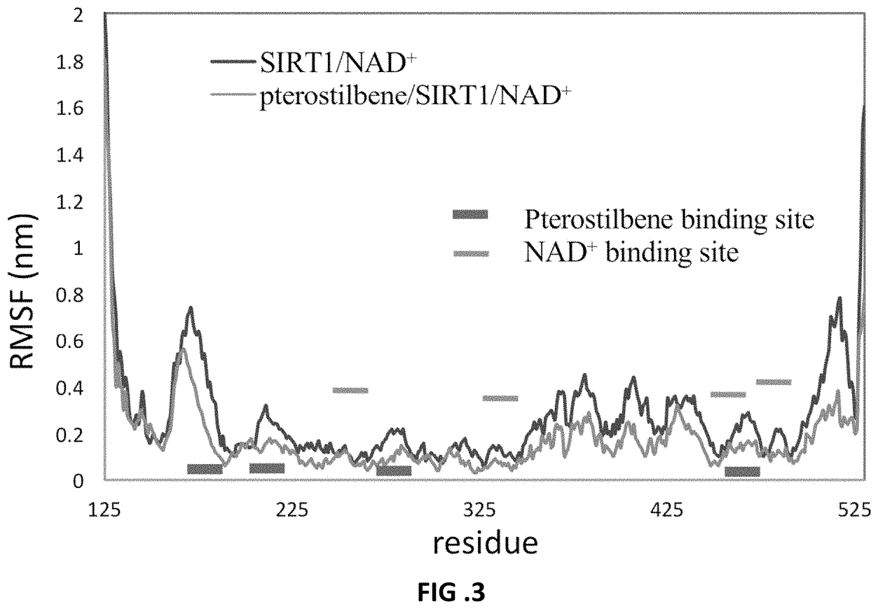

Next, the solvents were removed from the trajectory files, and Cα RMSD calculation (FIG. 2) and RMSF calculation (FIG. 3) were performed for both systems. FIG. 2 shows that the Cα RMSD values for both SIRT1/NAD⁺ complex and pterostilbene/SIRT1/NAD⁺ complex were lower than 1 nm within the 100 ns period, indicating that both systems were stable. Overall, the RMSD values of pterostilbene/SIRT1/NAD⁺ complex were slightly lower than those of the SIRT1/NAD⁺ complex, indicating that the addition of pterostilbene stabilized the SIRT1/NAD⁺ complex. FIG. 3 shows that the RMSF values of the pterostilbene binding sites on SIRT1 were lower than 1 nm without peaks, indicating that pterostilbene bound to SIRT1 stably. By comparing the RMSF values of the NAD⁺ binding sites on SIRT1 with and without pterostilbene, it is indicated that that the addition of pterostilbene stabilized the NAD⁺ binding on SIRT1.

Next, the 20 ns-100 ns trajectory from the 100 ns trajectory files for both systems were extracted, and 1 snapshot was taken at a frequency of every 8 ns to obtain a total of 10 snapshots for MM/GBSA (molecular mechanics/generalized Born surface area method. See Still, W. C., et al. “Semianalytical treatment of solvation for molecular mechanics and dynamics.” Journal of the American Chemical Society 112.16 (1990): 6127-6129) binding free energy (ΔG) calculation. ΔG of the SIRT1/NAD⁺ complex was determined at −52.46±3.63 kcal/mol, whereas ΔG of the pterostilbene/SIRT1/NAD⁺ complex was determined at −62.92±4.85 kcal/mol. By comparing ΔG of the two complex systems, it is indicated that pterostilbene strengthened the binding between NAD⁺ and SIRT1.

In conclusion, the above results indicate that pterostilbene is an effective SIRT1 activating compound.

### Example 2: Prediction of the Effectiveness of PQQ on NAD⁺/SIRT3 Binding

According to the above methods and procedures, experiments were carried out to predicte the effectiveness of PQQ on the binding between NAD⁺ and SIRT3. The results confirmed that PQQ is an effective SIRT3 activating compound. Detailed steps are described as follows.

The SIRT3 structural data was extracted from Protein Data Bank (PDB), i.e., 4FVT.pdb. Specifically, NAD⁺ analog carba-NAD⁺ and Ac-CS2 were removed to obtain the SIRT3 structural data. The PDB file of NAD⁺ was extracted from 4IF6.pdb. In addition, the partial charges were calculated and special structural information of PQQ molecule was obtained using Gaussian. All the structural information above were combined to create the structural input file from VMD.

Next, GOLD was used for the docking of NAD⁺ and PQQ onto SIRT3. Docking scores were obtained and compared. Both the SIRT3/NAD⁺ complex structure and PQQ/SIRT3/NAD⁺ complex structure were obtained.

Next, AmberTools was used to generate the topology files, prmtop files, and inperd files for the complexes, SIRT3, PQQ, and NAD⁺ in both the SIRT3/NAD⁺ complex structure and PQQ/SIRT3/NAD⁺ complex structure. Acpype.py was then used to convert the topology files to Gromacs formats. A temperature replica-exchange molecular dynamics simulation was then performed using the Amber99SB force field. All water bonds are constrained with SETTLE, and all other bonds are constrained with LINCS.

In Gromacs, a first round of energy minimization was performed to the SIRT3/NAD⁺ complex and PQQ/SIRT3/NAD⁺ complex via steepest descents until the maximum force is no greater than 1000 kJ/mol/nm. Then, the above systems were solvated in rectangular SPC/E water boxes. Na⁺ and Cl⁻ ions were added to achieve charge neutralization to get 0.155M NaCl-complex systems, and the solutes were required to be at least 1.2 nm away from the edges of the boxes. Afterwards, a first round of molecular dynamics simulation in canonical ensemble was performed, followed by a second round of energy minimization, with Velocity Rescale to heat the systems to and at 310 K for 10 ps (timestep=0.2 fs). Next, the second round of molecular dynamics simulation in canonical ensemble was performed for 100 ps (timestep=2 fs) and a molecular dynamics simulation in isothermal-isobaric ensemble was performed for 100 ps (timestep=2 fs) with Velocity Rescale for temperature control and Parinello-Rahman for pressure control, until temperature is stabilized at 310 K and pressure is stabilized at 1 atm, and the systems were fully equilibrated. Then, 20 replicas at different temperatures in the range of 310 K-400 K for both equilibrated complex systems were created, and a 100 ns temperature replica-exchange molecular dynamics simulation was performed to obtain the stable conformational structures corresponding to the lowest energy for both the SIRT3/NAD⁺ complex and PQQ/SIRT3/NAD⁺ complex with their trajectories.

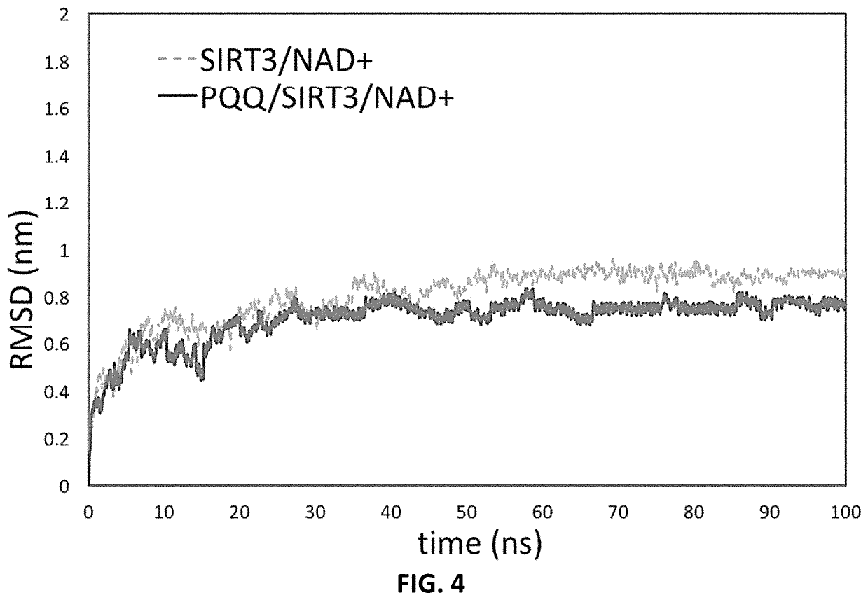

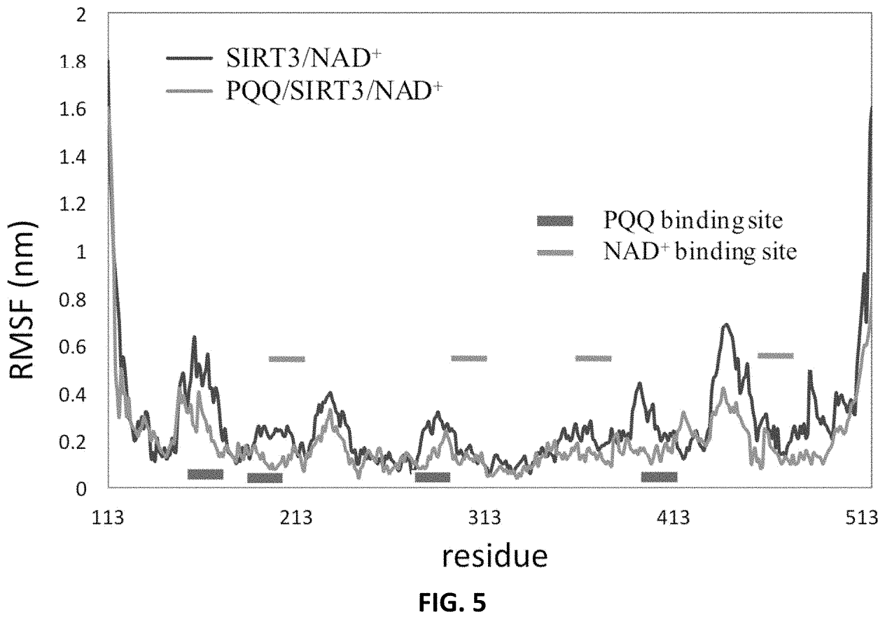

Next, the solvents were removed from the trajectory files, and Cα RMSD calculation (FIG. 4) and RMSF calculation (FIG. 5) were performed for both systems. FIG. 4 shows that the Cα RMSD values for both SIRT3/NAD⁺ complex and PQQ/SIRT3/NAD⁺ complex were lower than 1 nm within the 100 ns period, indicating that both systems were stable. Overall, the RMSD values of PQQ/SIRT3/NAD⁺ complex were slightly lower than those of SIRT3/NAD⁺ complex, indicating that the addition of PQQ stabilized the SIRT3/NAD⁺ complex. FIG. 5 shows that the RMSF values of the PQQ binding sites on SIRT3 were lower than 1 nm without peaks, indicating that PQQ bound to SIRT3 stably. By comparing the RMSF values of the NAD⁺ binding sites on SIRT3 with and without PQQ, it is indicated that the addition of PQQ stabilized the NAD⁺ binding on SIRT3.

Next, the 20 ns-100 ns trajectory from the 100 ns trajectory files for both systems were extracted, and 1 snapshot was taken at a frequency of every 8 ns to obtain a total of 10 snapshots for MM/GBSA binding free energy (ΔG) calculation. ΔG of SIRT3/NAD⁺ complex was determined at −63.17±4.35 kcal/mol, whereas ΔG of PQQ/SIRT3/NAD⁺ was determined at −82.23±5.24 kcal/mol. By comparing ΔG of the two complex systems, it is indicated that PQQ strengthened the binding between NAD⁺ and SIRT3.

In conclusion, the above results indicate that PQQ is an effective SIRT3 activating compound.

### Example 3: STAC Recipes Including NMN

Based on the performances and results as described herein, a new SYNFECT™ series STAC recipes that effectively acting on human sirtuins are designed. For example, in one of the recipes, the primary active ingredients are Nicotinamide Mononucleotide (NMN) and pterostilbene. In another recipe, the active ingredients are NMN and PQQ. In these STAC recipes, NMN be converted to NAD⁺ in vivo, thereby facilitating the formation of stable STAC/Sirtuin/NAD⁺ complexes.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of predicting or validating the effectiveness of a sirtuin-activating compounds (STAC) on the binding between NAD⁺ and a sirtuin protein, characterized in that the method comprises a replica-exchange molecular dynamics simulation, the method comprising:
(1) obtaining the structural data of a sirtuin protein from Protein Data Bank;
(2) generating the molecular structural input files for a STAC candidate and NAD⁺ using a molecular visualization software;
(3) docking the NAD⁺ to the corresponding binding pocket of the sirtuin protein to obtain a Sirtuin/NAD⁺ complex structure; docking the STAC candidate to the corresponding binding pocket of the Sirtuin/NAD⁺ complex structure to obtain a STAC/Sirtuin/NAD⁺ complex structure; and leaving the Sirtuin/NAD⁺ complex structure as a control;
(4) generating the topology files, prmtop files, and inperd files of the ligand, receptor, and complex system of both the Sirtuin/NAD⁺ complex and STAC/Sirtuin/NAD⁺ complex;
(5) converting the topology files in step (4) into Gromacs format;
(6) performing the replica-exchange molecular dynamics simulation on the two complex systems, comprising:
a) performing a first round of energy minimization to both systems, respectively,
b) solvating the systems and adding Na⁺ and Cl⁻ to achieve charge neutralization,
c) performing a first round of molecular dynamics simulation in canonical ensemble to the acquired solvated and charge neutralized systems,
d) performing a second round of energy minimization to both systems, respectively,
e) performing a second round of molecular dynamics simulation in canonical ensemble and a molecular dynamics simulation in isothermal-isobaric ensemble until the systems are fully equilibrated,
f) performing the replica-exchange molecular dynamics simulation to obtain equilibrated systems;
g) obtaining the stable conformation structures of both the Sirtuin/NAD⁺ and STAC/Sirtuin/NAD⁺ complexes from the free energy space minima, and
h) obtaining the corresponding trajectory files;
(7) removing the solvents from the trajectory files obtained in step (6); performing Cα RMSD calculation and RMSF calculation to determine if the STAC candidate stabilizes the Sirtuin/NAD⁺ complex;
(8) extracting snapshots at a frequency along the no-solvent trajectories from step (7), and performing binding free energy calculation between NAD⁺ and the sirtuin protein for both complexes to determine if the STAC candidate improves the binding between NAD⁺ and the sirtuin protein;
(9) predicting or validating the effect of the STAC candidate to the Sirtuin/NAD⁺ complex, according to the Cα RMSD, RMSF, and/or binding free energy changes observed in step (7) and step (8); and
(10) administering an effective amount of the STAC candidate to a subject in need thereof.

2. The method of claim 1, wherein the force field chosen for the replica-exchange molecular dynamics simulation in step (6) comprises any of AmberFF14SB, Amber99SB, gromacs54a7, GROMOS96, or GAFF.

3. The method of claim 1, wherein performing energy minimization to both complexes in step (6) comprises using steepest descents algorithm until the maximum force is no greater than 1000 kJ/mol/nm.

4. The method of claim 1, wherein the minimum distance between the solutes and the edge of the simulation box in step (6) is no less than 1 nm, and the water model is either SPC/E or TIP3P.

5. The method of claim 1, wherein the canonical ensemble molecular dynamics simulations in step (6) are performed under periodic boundary condition, and the first round of canonical ensemble molecular dynamics simulation further comprises of heating the systems to 300-320 K in less than 20 picosecond to release extra internal strain with a timestep less than 1 femtosecond; the second round of canonical ensemble molecular dynamics simulation further comprises of heating and running the system at 300-320 K for at least 50 picosecond with a timestep greater than 1 femtosecond.

6. The method of claim 1, wherein the isothermal-isobaric molecular dynamics simulation in step (6) is performed under periodic boundary condition, with temperature controlled to be 300-320 K and pressure controlled to be about 1 atm; and the systems are equilibrated for at least 50 picoseconds.

7. The method of claim 1, in step (6), the replica-exchange molecular dynamics simulation is a temperature replica-exchange molecular dynamics simulation, wherein the temperature is set to be 300-500K; or, the replica-exchange molecular dynamics simulation is a Hamilton replica-exchange molecular dynamics simulation, wherein the temperature is set to be a single value in the range of 300-500 K.

8. The method of claim 1, wherein in step (6), all water bonds are constrained with SETTLE, and all other bonds are constrained with LINCS; wherein a 1 nm cutoff is used for short range non-bonded interactions and Particle Mesh Ewald is used for long-range electrostatics.

9. The method of claim 1, wherein in step (8), the method of calculating binding free energy comprises using the prmtop files of ligand, receptor, and complex system for both complexes obtained in step (4), along with the no-solvent trajectory files, to perform MMPB(GB) SA calculation.

10. The method of claim 1, wherein predicting or validating the effect of the STAC candidate to the Sirtuin/NAD+ complex in step (9) further comprises:
evaluating the RMSD calculation results obtained in step (7), if the overall RMSD of the STAC/Sirtuin/NAD⁺ complex is smaller than 1 nm and is smaller than the overall RMSD of the Sirtuin/NAD⁺ complex, the STAC candidate stabilizes the Sirtuin/NAD⁺ complex;
evaluating the RMSF calculation results obtained in step (7), if the RMSF values of the binding site residues of the STAC candidate on the sirtuin protein are smaller than 1 nm, the binding site of the STAC candidate on the sirtuin protein is stable, if in the STAC/Sirtuin/NAD⁺ complex the RMSF values of the binding site residues of NAD⁺ on the sirtuin protein are smaller than 1 nm, the STAC candidate makes the binding between NAD⁺ and the sirtuin protein more stable;
evaluating the binding free energy calculation results obtained in step (8), if the binding free energy ΔG in the STAC/Sirtuin/NAD⁺ complex is negative, and its absolute value is greater than that in the Sirtuin/NAD⁺ complex, adding the STAC candidate strengthens the binding between NAD⁺ and the sirtuin protein;
if adding the STAC candidate stabilizes the complex, stabilizes the NAD⁺ and sirtuin binding site, strengthens the binding between NAD⁺ and the sirtuin protein, and the STAC and sirtuin biding site is stable, the STAC candidate is an effective STAC.

11. The method of claim 1, wherein the STAC candidate is selected from the group consisting of flavonoids, phenolic acids, stilbenes, lignans.

12. The method of claim 1, wherein the STAC candidate is resveratrol, pterostilbene, hesperatin, naringenin, catechin, quercetin, fisetin, caffeic acid, pinoresinol, pyrroloquinoline quinone, pycnogenol, curcumin, or jaceosidin.

13. The method of claim 1, wherein the sirtuin protein is from human SIRT1, SIRT2, SIRT3, SIRT4, SIRT5, SIRT6, and SIRT7.

14. A method of predicting or validating the effectiveness of a sirtuin-activating compounds (STAC) on the binding between NAD⁺ and a sirtuin protein, wherein the method comprises a replica-exchange molecular dynamics simulation, the method comprising:
(1) obtaining structural data of a sirtuin protein;
(2) generating a molecular structure for a STAC candidate and NAD⁺;
(3) docking the NAD⁺ to the corresponding binding pocket of the sirtuin protein to obtain a Sirtuin/NAD⁺ complex structure; docking the STAC candidate to the corresponding binding pocket of the Sirtuin/NAD⁺ complex structure to obtain a STAC/Sirtuin/NAD⁺ complex structure; and leaving the Sirtuin/NAD⁺ complex structure as a control;
(4) generating complex systems of both the Sirtuin/NAD⁺ complex and STAC/Sirtuin/NAD⁺ complex;
(5) performing the replica-exchange molecular dynamics simulation on the two complex systems, comprising:
a) performing a first round of energy minimization to both systems, respectively;
b) solvating the systems and adding Na⁺ and Cl⁻ to achieve charge neutralization;
c) performing a first round of molecular dynamics simulation in canonical ensemble to the acquired solvated and charge neutralized systems;
d) performing a second round of energy minimization to both systems, respectively;
e) performing a second round of molecular dynamics simulation in canonical ensemble and a molecular dynamics simulation in isothermal-isobaric ensemble until the systems are fully equilibrated;
f) performing the replica-exchange molecular dynamics simulation to obtain equilibrated systems;
g) obtaining the stable conformation structures of both the Sirtuin/NAD⁺ and STAC/Sirtuin/NAD⁺ complexes from the free energy space minima;
(6) performing Cα RMSD calculation and RMSF calculation to determine if the STAC candidate stabilizes the Sirtuin/NAD⁺ complex;
(7) extracting snapshots at a frequency along the no-solvent trajectories, and performing binding free energy calculation between NAD⁺ and the sirtuin protein for both complexes to determine if the STAC candidate improves the binding between NAD⁺ and the sirtuin protein; and
(8) predicting or validating the effect of the STAC candidate to the Sirtuin/NAD⁺ complex, according to the Cα RMSD, RMSF, and/or binding free energy changes.

15. The method of claim 14, wherein the method further comprises performing one or more experiments for testing effectiveness of a sirtuin-activating compounds (STAC) on the binding between NAD⁺ and a sirtuin protein.

16. The method of claim 14, wherein the method further comprises administering an effective amount of the STAC candidate to a subject in need thereof.
